(19) United States
(12) Patent Application Publication
(10) Pub. No.: US 2025/0073453 A1
(43) Pub. Date: Mar. 6, 2025

(54) SYSTEM FOR FUNCTIONAL ELECTRICAL STIMULATION AND ELECTROMYOGRAPHY MEASUREMENT

(71) Applicant: Battelle Memorial Institute, Columbus, OH (US)

(72) Inventors: Nicholas Annetta, Columbus, OH (US); Joshua R. Branch, Columbus, OH (US); Adam Farwick, Columbus, OH (US); Thomas Krenelka, Columbus, OH (US); Albert E. Weller, III, Marietta, GA (US); Jesse Keckler, Columbus, OH (US)

(21) Appl. No.: 18/611,827

(22) Filed: Mar. 21, 2024

Related U.S. Application Data
(60) Provisional application No. 63/535,939, filed on Aug. 31, 2023.

(51) Int. Cl. A61N 1/04; A61N 1/36

(52) U.S. Cl. CPC A61N 1/0484; A61N 1/0452; A61N 1/36031; A61N 1/0456

(57) ABSTRACT

A system for combined electromyography (EMG) measurements and stimulation of a target anatomy includes a garment with electrodes. An analog cable connects with the electrodes of the garment. A stimulator inputs electrical energy to the analog cable to deliver transcutaneous electrical stimulation. Stimulator isolation circuitry switches between connecting the stimulator with the analog cable and electrically disconnecting the stimulator from the analog cable. EMG circuitry receives analog EMG signals from the analog cable and forms digitized EMG data. EMG isolation circuitry switches between electrically connecting the EMG circuitry with the analog cable and electrically disconnecting the EMG circuitry from the analog cable. Control circuitry cyclically switches between stimulation and EMG time intervals. Clamping transistors connect between the EMG isolation circuitry and the EMG circuitry and cyclically switch between clamped time intervals in which the clamping transistors connect the clamped circuit nodes to a clamp voltage, and unclamped time intervals.

This application claims the benefit of U.S. Provisional Application No. 63/535,939 filed Aug. 31, 2023, which is incorporated herein by reference in its entirety.

### BACKGROUND

The following relates to the wearable electronic garment arts, transcutaneous functional electrical stimulation (FES) or neuromuscular electrical stimulation (NMES), transcutaneous electrical nerve stimulation (TENS), electromyography (EMG) measurements, providing for combined EMG measurements and FES, NMES, and/or TENS stimulation, and to the like.

Friedenberg et al., U.S. Pub. No. 2021/0379372 A1 published Dec. 9, 2021 and Blum et al., U.S. Pub. No. 2023/1191115 A1 published Jun. 22, 2023 present certain embodiments of systems for combined EMG measurements and FES, NMES, and/or TENS stimulation. Each of U.S. Pub. No. 2021/0379372 A1 and U.S. Pub. No. 2023/1191115 A1 is incorporated herein by reference in its entirety.

Certain improvements are disclosed herein.

### BRIEF SUMMARY

In accordance with some illustrative embodiments disclosed herein, a system is disclosed for combined electromyography (EMG) measurements of a target anatomy and stimulation of the target anatomy. The system comprises: a garment configured to be worn on the target anatomy and including electrodes arranged to electrically contact skin of the target anatomy when the garment is worn on the target anatomy and an analog cable comprising a bundle of electrical conductors connected with the electrodes of the garment; a stimulator configured to input electrical energy to a distal end of the analog cable to deliver transcutaneous electrical stimulation to the target anatomy via the electrodes of the garment; stimulator isolation circuitry configured to switch between electrically connecting the stimulator with the distal end of the analog cable and electrically disconnecting the stimulator from the distal end of the analog cable; EMG circuitry configured to receive analog EMG signals from the distal end of the analog cable and including analog amplifiers configured to amplify the analog EMG signals to form amplified analog EMG signals and analog-to-digital converters configured to digitize the amplified analog EMG signals to form digitized EMG data; EMG isolation circuitry configured to switch between electrically connecting the EMG circuitry with the distal end of the analog cable and electrically disconnecting the EMG circuitry from the distal end of the analog cable; and control circuitry. The control circuitry is configured to cyclically switch between: (i) stimulation time intervals during which the stimulator isolation circuitry electrically connects the stimulator with the distal end of the analog cable and the EMG isolation circuitry electrically disconnects the EMG circuitry from the distal end of the analog cable, and (ii) EMG time intervals during which the stimulator isolation circuitry electrically disconnects the stimulator from the distal end of the analog cable and the EMG isolation circuitry electrically connects the EMG circuitry with the distal end of the analog cable.

In accordance with some illustrative embodiments disclosed herein, a method of combined EMG measurements of a target anatomy and stimulation of the target anatomy is disclosed. The method comprises: during a stimulation time interval and using a stimulator, delivering transcutaneous electrical stimulation to the target anatomy via electrodes of a garment worn on the target anatomy; during an output time subinterval of an EMG time interval and using EMG circuitry, receiving analog EMG signals from the target anatomy via the electrodes of the garment and digitizing the analog EMG signals to form digitized EMG data; during the stimulation time interval and using EMG isolation circuitry, electrically isolating the EMG circuitry from the electrodes of the garment; during the EMG time interval and using stimulator isolation circuitry, electrically isolating the stimulator from the electrodes of the garment; except during the output time subintervals of the EMG time intervals, clamping circuit nodes located between the EMG isolation circuitry and the EMG circuitry to a clamp voltage; and during the output time subintervals of the EMG time intervals, unclamping the circuit nodes from the clamp voltage.

In accordance with some illustrative embodiments disclosed herein, a system for combined EMG measurements of a target anatomy and stimulation of the target anatomy is disclosed. The system comprises: a garment configured to be worn on the target anatomy and including electrodes arranged to electrically contact skin of the target anatomy when the garment is worn on the target anatomy; a stimulator configured to deliver transcutaneous electrical stimulation to the target anatomy via the electrodes of the garment; stimulator isolation circuitry configured to switch between electrically connecting the stimulator with the electrodes of the garment and electrically disconnecting the stimulator from the electrodes of the garment; EMG circuitry configured to receive analog EMG signals from the electrodes of the garment and including analog amplifiers configured to amplify the analog EMG signals to form amplified analog EMG signals and analog-to-digital converters configured to digitize the amplified analog EMG signals to form digitized EMG data; EMG isolation circuitry configured to switch between electrically connecting the EMG circuitry with the electrodes of the garment and electrically disconnecting the EMG circuitry from the electrodes of the garment; and control circuitry. The control circuitry is configured to cyclically switch between: (i) stimulation time intervals during which the stimulator isolation circuitry electrically connects the stimulator with the electrodes of the garment and the EMG isolation circuitry electrically disconnects the EMG circuitry from the electrodes of the garment, and (ii) EMG time intervals during which the stimulator isolation circuitry electrically disconnects the stimulator from the electrodes of the garment and the EMG isolation circuitry electrically connects the EMG circuitry with the electrodes of the garment. The system further includes clamping transistors electrically connected at circuit nodes located between the EMG isolation circuitry and the EMG circuitry. The control circuitry is further configured to cyclically switch between clamped time intervals in which the clamping transistors are conductive to connect the clamped circuit nodes to a clamp voltage and unclamped time intervals in which the clamping transistors are nonconductive. Each clamped time interval is a contiguous time interval that includes a corresponding stimulation time interval and a recovery time subinterval of an EMG time interval immediately following the corresponding stimulation time interval.

In accordance with some illustrative embodiments disclosed herein, a system for combined EMG measurements and stimulation of a target anatomy includes a garment with electrodes. An analog cable connects with the electrodes of the garment. A stimulator inputs electrical energy to the analog cable to deliver transcutaneous electrical stimulation. Stimulator isolation circuitry switches between connecting the stimulator with the analog cable and electrically disconnecting the stimulator from the analog cable. EMG circuitry receives analog EMG signals from the analog cable and forms digitized EMG data. EMG isolation circuitry switches between electrically connecting the EMG circuitry with the analog cable and electrically disconnecting the EMG circuitry from the analog cable. Control circuitry cyclically switches between stimulation and EMG time intervals. Clamping transistors connect between the EMG isolation circuitry and the EMG circuitry and cyclically switch between clamped time intervals in which the clamping transistors connect the clamped circuit nodes to a clamp voltage, and unclamped time intervals.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically illustrates a block diagram of a system for combined EMG measurements and FES, NMES, and/or TENS stimulation.

FIG. 2 diagrammatically illustrates a schematic electrical diagram of an embodiment of the system of FIG. 1.

FIG. 3 diagrammatically illustrates an embodiment of the analog EMG readout chain of the system of FIG. 1.

FIG. 4 diagrammatically illustrates a timing diagram of operation of the system of FIG. 1.

### DETAILED DESCRIPTION

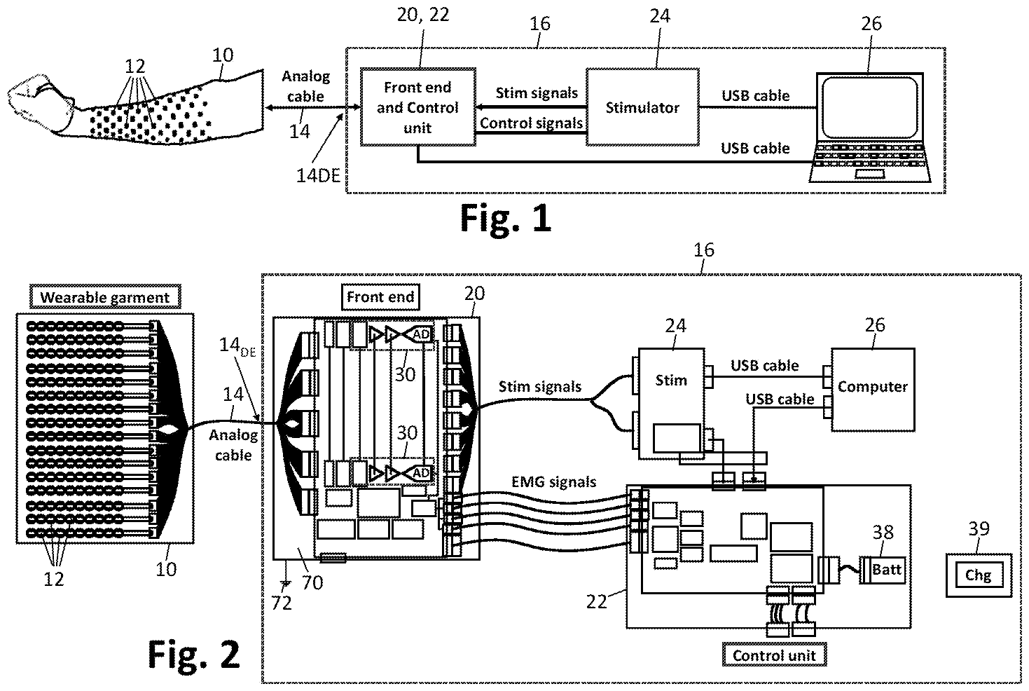

With reference to FIG. 1, a block diagram is shown of a system for combined electromyography (EMG) measurements of a target anatomy and transcutaneous electrical stimulation of the target anatomy. The transcutaneous electrical stimulation may, for example, comprise functional electrical stimulation (FES) that produces movement of the target anatomy, or neuromuscular electrical stimulation (NMES) of the anatomy, and/or transcutaneous electrical nerve stimulation (TENS) of nerves of the anatomy. As used herein, “EMG” is to be broadly construed as encompassing electromyography signals and also neural signals generated by nerves of the target anatomy. The system of FIG. 1 includes a garment 10, such as an illustrative sleeve worn on an arm. In this case, the target anatomy is an arm. The garment 10 could more generally be a garment worn on another target anatomy, such as a leg, a torso, or so forth. Moreover, while the illustrative sleeve 10 extends over the forearm and wrist, in other embodiments the sleeve may only cover a portion of that anatomy. In general, the garment 10 covers or is worn on the target anatomy from which EMG measurements are to be acquired and to which transcutaneous electrical stimulation is to be applied. As diagrammatically shown in FIG. 1, the garment 10 includes electrodes 12 distributed over all or that portion of the target anatomy from which EMG is to be measured and to which transcutaneous electrical stimulation is to be applied. FIG. 1 is to be understood as diagrammatic, as the electrodes 12 are disposed on the inside surface of the garment to provide transcutaneous contact with nerves and/or muscles of the target anatomy (such that the electrodes are typically not visible when the garment 10 is worn on the target anatomy. The electrodes 12 are thus transcutaneous electrodes, insofar as they measure EMG through the skin of the target anatomy and apply transcutaneous electrical stimulation, that is, stimulation through the skin.

The garment 10 is designed to fit snugly onto the target anatomy to provide good electrical contact between the electrodes 12 and the skin of the target anatomy. For example, the garment 10 can be implemented as a compression sleeve to advantageously compress the electrodes against the skin, or may include inflatable bladders to press the electrodes 12 against the skin, or so forth. For example, in some embodiments the illustrative sleeve 10 comprises an elastane fabric such as spandex or lycra. Elastane fabrics comprise fibers of a long chain polyurethane, e.g. a polyether-polyurea copolymer. To provide good electrical conductivity with the skin, the electrodes 12 can by way of nonlimiting illustrative example comprise hydrogel discs, metal (e.g. steel) discs plated with an electrically conductive metal such as gold, palladium, or silver, or may comprise a compressible polymer and a conductive filler dispersed in the compressible polymer. The conductive filler may be, e.g., carbon fibers, carbon nanotubes (CNTs), or metallic particles. The foregoing are merely nonlimiting illustrative examples of some suitable garment 10 and electrode 12 designs.

With continuing reference to FIG. 1, the electrodes 12 of the garment 10 are connected by way of an analog cable 14 with electronics 16 that provide for measurement of EMG generated in the target anatomy using the electrodes 12 and also for applying transcutaneous electrical stimulation to the target anatomy using the electrodes 12. Notably, in the illustrative embodiments herein, the electronics 16 are entirely external of the garment 10, and analog EMG signals received at the electrodes 12 are transmitted to the electronics 16 via the analog cable 14; and likewise analog transcutaneous electrical stimulation pulses are transmitted to the electrodes 12 via the analog cable 14. The analog cable 14 comprises a bundle of twisted wire pairs, coaxial cables, triaxial cables, or the like, with each pair of electrodes of the electrodes 12 used for EMG readout and transcutaneous electrical stimulation being connected to the electronics 16 via a corresponding twisted wire pair or coaxial or triaxial cable of the analog cable 14.

With continuing reference to FIG. 1, the illustrative electronics 16 are implemented as three physical modules: a front end and control unit 20, 22; a stimulator 24 that generates the transcutaneous electrical stimulation pulses for FES, NMES, and/or TENS; and a computer 26 (or other digital processing device 26) that controls the other modules 20, 22, and 24 to perform useful tasks employing a combination of EMG measurement from the target anatomy and transcutaneous electrical stimulation of the target anatomy. The analog cable 14 is attached to the garment 10 and has a distal end 14DE that connects with the front end/control unit 20, 22.

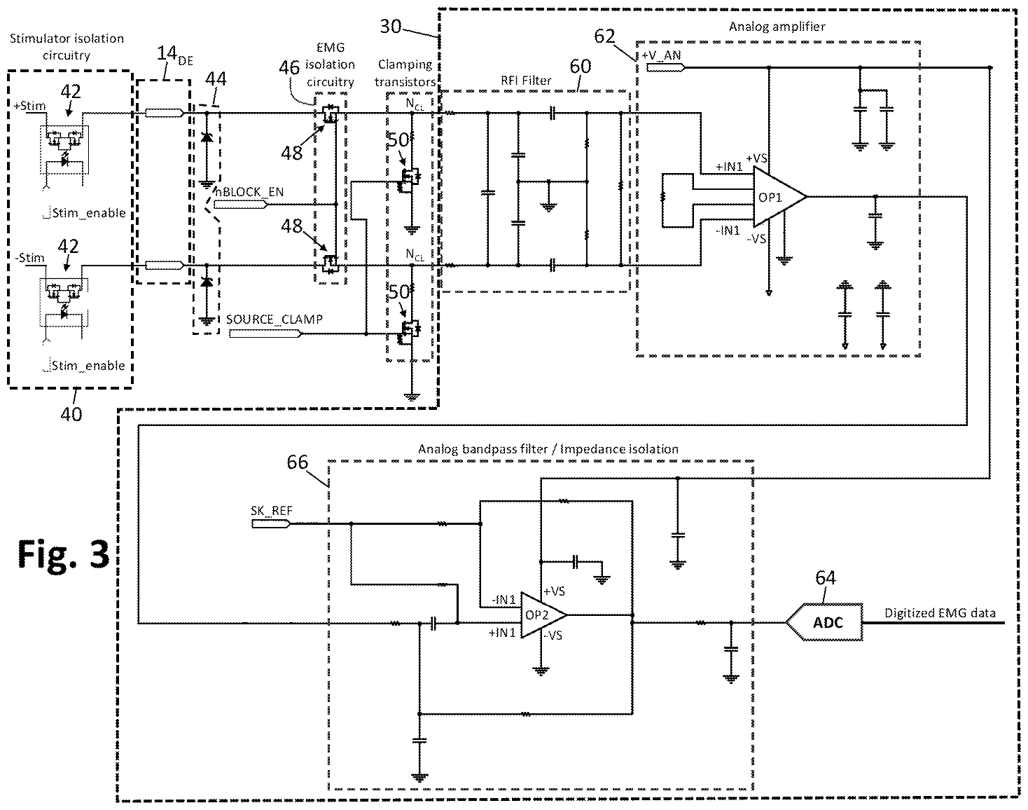

With continuing reference to FIG. 1 and with further reference to FIG. 2 which diagrammatically illustrates a schematic electrical diagram of an embodiment of the system of FIG. 1, the stimulator 24 is connected with the front end 20 to input electrical energy to the distal end 14DE of the analog cable 14 to deliver transcutaneous electrical stimulation to the target anatomy via the electrodes 12 of the garment 10. The front end 20 includes EMG circuitry 30 for receiving, amplifying, and digitizing analog EMG signals from the distal end 14DE of the analog cable 14 to form digitized EMG data, and other components and/or circuitry for providing switching between and electrical isolation for EMG measurement and transcutaneous electrical stimulation. An illustrative example of suitable electric circuitry for such functionality will be described in more detail with reference to FIG. 3. The example of FIG. 2 divides the front end and control unit 20, 22 into a front end 20 that includes the EMG circuitry 30, and a control unit 22 that includes the control circuitry-however, in some embodiments these may be disposed in a single module, for example disposed on a single printed circuit board (as diagrammatically indicated in FIG. 1). In addition to the analog cable 14 connecting the electrodes 12 of the garment 10 with the front end/control unit 20, 22, the illustrative design includes stimulation cables carrying analog stimulation signals (i.e., electrical energy) from the stimulator 24 to the front end 20, and various digital data and/or control cables (e.g., USB cables in some illustrative examples of FIG. 1) for conveying control and/or data between the various units. As shown in FIG. 2, the system may include additional components not described in detail herein, such as an illustrative rechargeable battery 38 or other power supply and a battery charger 39 in the form of a plug receptacle, wireless inductive battery charger, or the like for recharging the battery 38.

The illustrative design of FIGS. 1 and 2 places all electronics (other than the electrodes 12 and wiring, flexible printed circuit boards, or other electrical conductors connecting the electrodes 12 with the analog cable 14) off the garment 10. In the illustrative example, the stimulator 24 is not mounted on or in the garment 10, and the EMG circuitry and the stimulator isolation circuitry and EMG isolation circuitry (to be described) are also not mounted on or in the garment 10, and still further the control circuitry 22 is not mounted on or in the garment 10. This advantageously makes the sleeve or other garment 10 lighter-weight, cooler (by removing the heat-generating electronics from the garment 10), and more comfortable for the wearer of the garment 10.

However, implementing the arrangement of FIGS. 1 and 2 presents certain challenges. Notably, the placement of the EMG circuitry 30 on the front end 20 connected at the distal end 14DE of the analog cable 14 means that the analog EMG signals are conducted a relatively long distance (about the length of the analog cable 14) before reaching the EMG circuitry 30 which provides the EMG signal amplification and digitization. By contrast, some embodiments disclosed in Friedenberg et al., U.S. Pub. No. 2021/0379372 A1 and Blum et al., U.S. Pub. No. 2023/1191115 A1 integrate at least the EMG amplifiers into the sleeve or other garment, which minimizes the signal path length the analog EMG signals travel before amplification/digitization. Even if the analog cable 14 of the embodiment of FIGS. 1 and 2 is well shielded, this long path length for the analog EMG signals can introduce signal degradation and signal loss so that the (typically low-amplitude) analog EMG signals could have lower signal-to-noise ratio (SNR) compared with on-sleeve EMG signal processing designs.

One way to reduce the signal loss over the analog cable 14 would be to make the analog cable 14 short. However, this can be ergonomically unsatisfactory, as it would place the front end 20 close to the garment 10, which could impede mobility of the arm or other target anatomy wearing the garment 10. For the illustrative example in which the garment 10 is a sleeve worn on target anatomy comprising an arm, a short length for the analog cable 14 would likely impede movement of the elbow and/or arm.

Another challenge is to minimize interference between the stimulation and EMG measurement phases of a combined EMG/transcutaneous electrical stimulation operating flow. Notably, the amount of electrical energy input to the distal end 14DE of the analog cable 14 during transcutaneous electrical stimulation is much higher than the electrical energy produced by the muscles and/or nerves of the target anatomy which constitute the analog EMG signals. Hence, it is possible for the high energy stimulation to produce artifacts in the EMG measurement. In embodiments disclosed herein, the same electrodes 12 are used for both EMG signal measurement and transcutaneous electrical stimulation. To do so, the stimulation and EMG are time domain multiplexed (TDM); that is, during transcutaneous electrical stimulation the EMG is not measured, and conversely during EMG measurement transcutaneous electrical stimulation is not applied. In the TDM, a recovery period (T_{R}) is interposed between the end of the transcutaneous electrical stimulation and the start of EMG measurement. This recovery period allows for electrical charge built up in the electrodes 12, analog cable 14, and in the electronics 16 during the stimulation phase to dissipate before initiating the EMG measurement. The analog cable 14 can introduce additional capacitance and charge accumulation that should be dissipated before initiation of the EMG measurement (and hence entails a longer recovery period). However, for smooth functioning of tasks employing combined EMG and transcutaneous electrical stimulation it is often desirable for the recovery period to be as short as practicable. For example, in one application for assisting a patient impaired by stroke, spinal injury, or the like, the EMG signals are measured to determine the user's intent (e.g., which muscles the user wants to utilize) and the transcutaneous electrical stimulation then performs functional electrical stimulation (FES) of those muscles to cause the target anatomy to perform the motion desired by the user. For this application, rapid switching between EMG measurement and stimulation is desired to simulate natural thought-guided anatomical motion.

In illustrative embodiments described herein, the electronics 16 incorporate certain features to address these considerations and enable the system for combined EMG measurements of the target anatomy and transcutaneous electrical stimulation of the target anatomy to be performed with high switching speed between the stimulation and EMG measurement phases.

With reference now to FIG. 3, an embodiment of the analog EMG readout chain of the system of FIG. 1 is shown by way of an electrical schematic. FIG. 3 also diagrammatically illustrates the distal end 14DE of the analog cable 14 represented as electrical connections to a pair of the electrodes 12 of the garment 10, and stimulator isolation circuitry 40 comprising solid state relays 42 for isolating the stimulator 24 (shown in FIGS. 1 and 2) during the EMG phase, electrostatic discharge (ESD) protection circuitry 44, EMG isolation circuitry 46 comprising blocking transistors 48, and clamping transistors 50. It is to be understood that FIG. 3 illustrates an electrical schematic this circuitry 30, 40, 44, 46, 50 for a single pair of electrodes of the set of electrodes 12 of the garment 10. Without loss of generality, if there are N electrode pairs for the EMG measurements/stimulation, then there will be N instances of the circuitry 30, 40, 44, 46, 50 shown in FIG. 3 (one instance for each electrode pair). Put another way, FIG. 3 illustrates the electrical schematic for one channel of the EMG circuitry.

The stimulator isolation circuitry 40 includes the illustrative solid state relays 42. The control circuitry 22 is configured to close the solid state relays 42 during each stimulation time interval to connect the stimulator 24 with the electrodes 12 to enable application of transcutaneous electrical stimulation to the target anatomy. The control circuitry 22 is further configured to open the solid state relays 42 during each EMG time interval to electrically isolate the stimulator 24 from the electrodes 12. In one nonlimiting illustrative example, the solid state relays 42 may be G3VM-41QR10TR05 solid state relays (available from Omron Electronics, Inc.) which employ optical (light emitting diode, i.e., LED) actuators triggered by the Stim_enable signal indicated in FIG. 3. The solid state relays 42 are closed during a stimulation time interval (T_{STIM}), and the solid state relays 42 are open during an EMG time interval (T_{EMG}).

The EMG isolation circuitry 46 includes the illustrative blocking transistors 48. The control circuitry 22 is configured to close the blocking transistors 48 to electrically connect the EMG circuitry 30 with the distal end 14DE of the analog cable 14, and to open the blocking transistors 48 to electrically disconnect the EMG circuitry 30 from the distal end 14DE of the analog cable 14. The blocking transistors 48 are closed during each EMG time interval (T_{EMG}) to electrically connect the EMG circuitry 30 with the electrodes 12, and are open during the stimulation time interval (T_{STIM}) to block the simulation voltages applied to the distal end 14DE of the analog cable 14 from reaching the EMG circuitry 30. In some nonlimiting illustrative embodiments, the blocking transistors 48 are MOSFET devices, such as BSS127SSN-7 n-channel MOSFET devices available from Diodes Incorporated, which are rated for 600 V and 50 mA.

The ESD protection circuitry 44 in the illustrative example comprises Zener diodes, such as SMF200A TVS diodes available from Littlefuse Inc and rated for 200V. The ESD protection circuitry 44 is optional but beneficial to protect the EMG circuitry 30 against electrostatic discharge events.

With continuing reference to FIG. 3, the illustrative EMG circuitry 30 (for each channel, one of which is illustrated in FIG. 3) includes a radio frequency interference (RFI) filter 60, an analog amplifier 62, an analog-to-digital converter (ADC) 64, and an analog bandpass filter 66 interposed between the output of the analog amplifier 62 and the input of the ADC 64. Aspects of these components of the EMG circuitry 30 will be described in further detail later herein.

As noted above, the EMG isolation circuitry 46 including the blocking transistors 48 provide isolation for the EMG circuitry 30 during the stimulation time interval (T_{STIM}). This might be expected to be sufficient to provide combined transcutaneous electrical stimulation to the target anatomy and time-domain multiplexed EMG measurement. However, it is recognized herein that the EMG isolation circuitry 46 may be insufficient to provide such time domain multiplexed operation, especially if high speed switching between the transcutaneous electrical stimulation and the EMG measurement is desired. This is in part due to the use of the analog cable 14 connecting the electrodes 12 of the garment 10 with the electronics 16 (see FIGS. 1 and 2). As previously noted, the analog cable 14 can introduce additional capacitance and charge accumulation during the stimulation time interval (T_{STIM}) that should be dissipated before initiation of the EMG measurement. The recovery period (T_{R}) accommodates this transition, but for smooth functioning of tasks employing combined EMG and transcutaneous electrical stimulation it is desirable for T_{R} to be as short as practicable to provide rapid switching between stimulation and EMG measurement.

To this end, as shown in FIG. 3 the clamping transistors 50 are electrically connected at circuit nodes N_{CL} located between respective blocking transistors 48 and the EMG circuitry. Again, FIG. 3 illustrates one channel corresponding to one pair of electrodes. The control circuitry 22 is further configured to cyclically switch between clamped time intervals in which the clamping transistors 50 are conductive to connect the clamped circuit nodes N_{CL} to a clamp voltage (SOURCE_CLAMP), and unclamped time intervals in which the clamping transistors are nonconductive to disconnect the circuit nodes N_{CL} from the clamp voltage. In some embodiments, the clamp voltage is electrical ground. In some embodiments, the clamp voltage (SOURCE_CLAMP) is a negative voltage. In some nonlimiting illustrative examples, the clamping transistors 50 are CMUDM7005 n-channel MOSFET devices available from Central Semiconductor Corp.

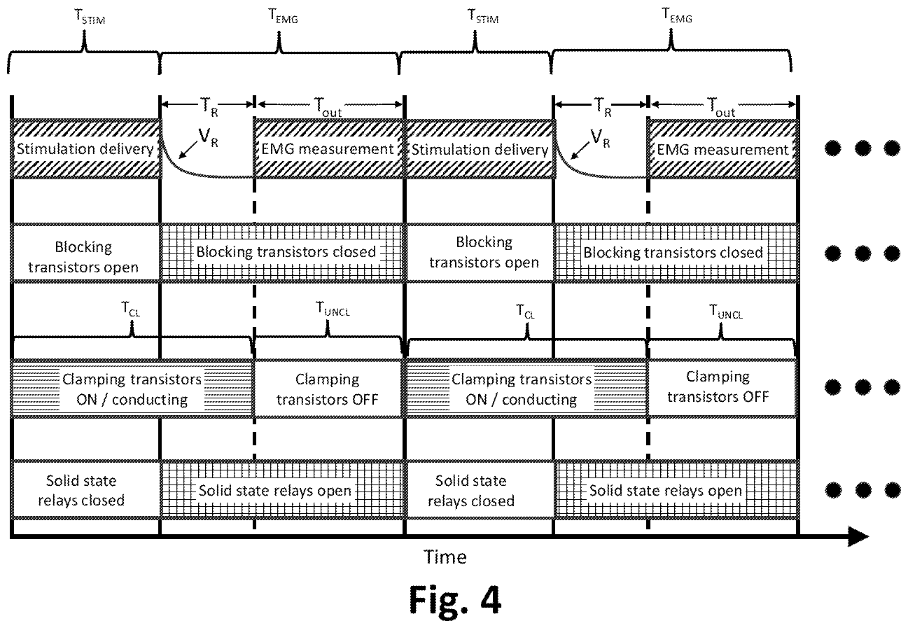

With continuing reference to FIG. 3 and with further reference to FIG. 4, the latter FIG. 4 presents a timing diagram for time-domain multiplexed cyclic switching between transcutaneous electrical stimulation using the stimulator 24 and EMG measurement using the EMG circuitry 30. Indicated in FIG. 1 are two periods of this successive cycling, including a stimulation time interval (T_{STIM}), an EMG interval (T_{EMG}), a second stimulation time interval (T_{STIM}), and a second EMG interval (T_{EMG}). It will be appreciated that this cycling can continue, as controlled by the control circuitry 22, for the duration of the task employing both stimulation and EMG measurement. As indicated in FIG. 4, during each stimulation time interval (T_{STIM}) the blocking transistors 48 are open to isolate the EMG circuitry 30 from the electrodes, while the solid state relays 42 are closed to connect the stimulator 24 with the electrodes. Conversely, during each EMG time interval (T_{EMG}) the blocking transistors 48 are closed to connect the EMG circuitry 30 with the electrodes, while the solid state relays 42 are open to isolate the stimulator 24 from the electrodes.

Additionally, however, as seen in FIG. 4 the clamping transistors 50 cyclically switch between clamped time intervals (T_{CL}) in which the clamping transistors 50 are conductive to connect the clamped circuit nodes N_{CL} to the clamp voltage, and unclamped time intervals (T_{UNCL}) in which the clamping transistors 50 are nonconductive to disconnect the clamped circuit nodes N_{CL} from the clamp voltage. In general, the clamping transistors should be on (conductive) at least during the recovery time (T_{R}) to provide rapid discharge of capacitance charge and/or other charge accumulation on the analog cable 14 and/or electrodes 12 or in other components of the circuitry 16. This recovery is diagrammatically shown in FIG. 4 as a voltage VR (or, equivalently, a charge) that is being dissipated during the recovery time (T_{R}) at least in part via the path from the clamped circuit nodes N_{CL} to the clamp voltage (electrical ground or negative clamp voltage SOURCE_CLAMP indicated in FIG. 3, depending on the choice for the clamp voltage). In the illustrative example, the clamped time intervals T_{CL} are contiguous time intervals that extend over each stimulation time interval (T_{STIM}) and the recovery time (sub-) interval of the following EMG time interval (T_{EMG}). That is, the clamped time intervals (T_{CL}) are contiguous time intervals coinciding with the stimulation time intervals (T_{STIM}) and recovery time subintervals (T_{R}) of the EMG time intervals (T_{EMG}) which immediately follow preceding stimulation time intervals.

By extending the clamped time intervals (T_{CL}) to encompass the stimulation time intervals (T_{STIM}), the clamping also provides further protection for the EMG circuitry 30 during the transcutaneous electrical stimulation, and serves to continuously dissipate any electric charge that leaks through the open blocking transistors 48 thereby further reducing the requisite recovery time subinterval (T_{R}).

A further advantage of extending the clamped time intervals (T_{CL}) to encompass the stimulation time intervals (T_{STIM}) is that the conducting clamping transistors 50 during stimulation provide a safety function. In the event of a failure of isolation, such as if for example the blocking transistors 48 fail to open during the stimulation time interval (T_{STIM}) so that the electrical stimulation is not blocked from the EMG circuitry 30, the conducting clamping transistors 50 provide a path to electrical ground (or to a negative clamping voltage SOURCE_CLAMP) which prevents the voltage reaching the EMG circuitry 30 from rising above a limiting voltage across the conducting clamping transistors 50. For example, when using CMUDM7005 n-channel MOSFET devices as the clamping transistors 50, the maximum voltage reaching the EMG circuitry 30 is expected to be about 5 volts (whereas, the voltage on the electrodes pair for applying transcutaneous electrical stimulation to the target anatomy may be in excess of 100 volts).

The unclamped time intervals (T_{UNCL}) are then contiguous time intervals coinciding with output time subintervals (Tour indicated in FIG. 4) of the EMG time intervals during which the EMG circuitry is operating to form the digitized EMG data. It should be noted that the EMG circuitry may optionally be outputting data during the recovery time subintervals (T_{R}) and/or the stimulation time intervals (T_{STIM}) as well, but such data is expected to be noisy (during T_{R}) or invalid (during T_{STIM} since the EMG circuitry is isolated from the electrodes during T_{STIM}) and is suitably discarded. Conversely, digitized EMG data may optionally be formed and/or utilized over only a subinterval of output time subinterval (T_{OUT}).

By way of the clamping transistors 50, the recovery time subinterval (T_{R}) can be short even in the face of the capacitance and potential charge accumulation in the extended analog signal path presented by the analog cable 14. In some nonlimiting illustrative embodiments, T_{R} has a duration of 4 milliseconds or less. In some nonlimiting illustrative embodiments, T_{R} has a duration of between 1 millisecond and 4 milliseconds. These are merely nonlimiting illustrative examples.

With returning focus on FIG. 3, the EMG circuitry 30 is further described. The RFI filter 60 is an optional component which suitably filters out signal components at frequencies known or expected to contain RFI and not expected to contain useful EMG measurement information. The illustrative RFI filter 60 is a passive RC-based filter, but other types of filters are contemplated. The illustrative analog amplifier 62 is an operational amplifier (op amp) based analog amplifier. The illustrative analog amplifier 62 advantageously provides a high input impedance and a low output impedance. In one nonlimiting illustrative embodiment, the op amp of the analog amplifier 62 is an AD8224HBCPZ-WP instrument amplifier available from Analog Devices. The illustrative analog amplifier 62 may, for example, have a gain of 10, although the gain can be suitably adjusted using known analog amplifier tuning approaches. The output of the analog amplifier 62 is amplified but not filtered (other than the pre-filtering provided by the RFI filter 60).

In the illustrative EMG circuitry 30, the output of the analog amplifier 62 is input to the illustrative analog bandpass filter 66 which is interposed between the output of the analog amplifier 62 and the input of the ADC 64. The analog bandpass filter 66 may provide frequency filtering, and also serves as additional impedance isolation. In the illustrative embodiment, the analog bandpass filter 66 is an analog Sallen Key bandpass filter 66. However, other types of analog voltage-controlled voltage-source (VCVS) bandpass filters can be employed, or even more generally other types of analog bandpass filters with high (e.g., practically infinite) input impedance and low (e.g., nearly zero) output impedance can be employed.

With reference back to FIG. 2, in the illustrative embodiment the stimulator isolation circuitry 40, the EMG circuitry 30, and the EMG isolation circuitry 46 (as well as the clamping transistors 50 and the ESD protection 44) are disposed on a single printed circuit board 70 having a common electrical ground 72 for the EMG circuitry 30 and the input electrical energy input by the stimulator 24. The distal end 14DE of the analog cable 14 is connected with the single printed circuit board 70. The use of the common ground 72 further reduces noise and interference on the EMG measurement.

With reference back to FIGS. 1-3, in the illustrative example, a first module 20, 22 includes the stimulator isolation circuitry 40, the EMG circuitry 30, the EMG isolation circuitry 46, and the control circuitry 22, and the distal end 14DE of the analog cable 14 is connected with the first module 20, 22. The stimulator 24 is separate from the first module 20, 22 and is connected with the first module 20, 22 by one or more electrical cables (labeled “Stim signals” in FIGS. 1 and 2). The illustrative system further includes the computer 26 connected to control the first module 20, 22 and the stimulator 24 and to receive the digitized EMG data from the EMG circuitry 30. This is merely one illustrative layout, and the various components can be variously combined or separated.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A system for combined electromyography (EMG) measurements of a target anatomy and stimulation of the target anatomy, the system comprising:
a garment configured to be worn on the target anatomy and including electrodes arranged to electrically contact skin of the target anatomy when the garment is worn on the target anatomy and an analog cable comprising a bundle of electrical conductors connected with the electrodes of the garment;
a stimulator configured to input electrical energy to a distal end of the analog cable to deliver transcutaneous electrical stimulation to the target anatomy via the electrodes of the garment;
stimulator isolation circuitry configured to switch between electrically connecting the stimulator with the distal end of the analog cable and electrically disconnecting the stimulator from the distal end of the analog cable;
EMG circuitry configured to receive analog EMG signals from the distal end of the analog cable and including analog amplifiers configured to amplify the analog EMG signals to form amplified analog EMG signals and analog-to-digital converters configured to digitize the amplified analog EMG signals to form digitized EMG data;
EMG isolation circuitry configured to switch between electrically connecting the EMG circuitry with the distal end of the analog cable and electrically disconnecting the EMG circuitry from the distal end of the analog cable; and
control circuitry configured to cyclically switch between:
(i) stimulation time intervals during which the stimulator isolation circuitry electrically connects the stimulator with the distal end of the analog cable and the EMG isolation circuitry electrically disconnects the EMG circuitry from the distal end of the analog cable, and
(ii) EMG time intervals during which the stimulator isolation circuitry electrically disconnects the stimulator from the distal end of the analog cable and the EMG isolation circuitry electrically connects the EMG circuitry with the distal end of the analog cable.

2. The system of claim 1, wherein the EMG isolation circuitry includes blocking transistors configured to switch between electrically connecting the EMG circuitry with the distal end of the analog cable and electrically disconnecting the EMG circuitry from the distal end of the analog cable.

3. The system of claim 2, further comprising:
clamping transistors electrically connected at circuit nodes located between respective blocking transistors and the EMG circuitry;
wherein the control circuitry is further configured to cyclically switch between clamped time intervals in which the clamping transistors are conductive to connect the clamped circuit nodes to a clamp voltage and unclamped time intervals in which the clamping transistors are nonconductive.

4. The system of claim 3, wherein the clamp voltage is electrical ground.

5. The system of claim 3, wherein the clamp voltage is a negative voltage.

6. The system of claim 3, wherein:
the clamped time intervals are contiguous time intervals coinciding with the stimulation time intervals and recovery time subintervals of the EMG time intervals which immediately follow preceding stimulation time intervals, and
the unclamped time intervals are contiguous time intervals coinciding with output time subintervals of the EMG time intervals during which the EMG circuitry forms the digitized EMG data.

7. The system of claim 6, wherein the recovery time subintervals are of duration 4 milliseconds or less.

8. The system of claim 1, wherein:
the stimulator is not mounted on or in the garment,
the stimulator isolation circuitry is not mounted on or in the garment,
the EMG circuitry is not mounted on or in the garment,
the EMG isolation circuitry is not mounted on or in the garment, and
the control circuitry is not mounted on or in the garment.

9. The system of claim 1, wherein:
a first module includes the stimulator isolation circuitry, the EMG circuitry, the EMG isolation circuitry, and the control circuitry, the distal end of the analog cable connected with the first module;
the stimulator is separate from the first module and is connected with the first module by one or more electrical cables; and
the system further includes a computer connected to control the first module and the stimulator and to receive the digitized EMG data.

10. The system of claim 1, wherein:
the stimulator isolation circuitry, the EMG circuitry, and the EMG isolation circuitry are disposed on a single printed circuit board having a common ground for the EMG circuitry and the input electrical energy input by the stimulator; and
the distal end of the analog cable is connected with the single printed circuit board.

11. The system of claim 1, wherein the EMG circuitry further includes analog bandpass filters interposed between outputs of respective analog amplifiers and inputs of respective analog-to-digital converters.

12. The system of claim 11, wherein the analog bandpass filters are voltage-controlled voltage-source (VCVS) bandpass filters.

13. The system of claim 12, wherein the VCVS bandpass filters are Sallen Key bandpass filters.

14. The system of claim 1, wherein the analog cable comprises a bundle of twisted wire pairs in which each twisted wire pair connects to a pair of the electrodes of the garment.

15. A method of combined electromyography (EMG) measurements of a target anatomy and stimulation of the target anatomy, the method comprising:
during a stimulation time interval and using a stimulator, delivering transcutaneous electrical stimulation to the target anatomy via electrodes of a garment worn on the target anatomy;
during an output time subinterval of an EMG time interval and using EMG circuitry, receiving analog EMG signals from the target anatomy via the electrodes of the garment and digitizing the analog EMG signals to form digitized EMG data;
during the stimulation time interval and using EMG isolation circuitry, electrically isolating the EMG circuitry from the electrodes of the garment;
during the EMG time interval and using stimulator isolation circuitry, electrically isolating the stimulator from the electrodes of the garment;
except during the output time subintervals of the EMG time intervals, clamping circuit nodes located between the EMG isolation circuitry and the EMG circuitry to a clamp voltage; and
during the output time subintervals of the EMG time intervals, unclamping the circuit nodes from the clamp voltage.

16. The method of claim 15, wherein the clamp voltage is electrical ground.

17. The method of claim 15, wherein the clamp voltage is a negative voltage.

18. The method of claim 15, wherein the clamping is maintained after each stimulation time interval for a recovery time subinterval of the succeeding EMG time interval, the recovery time subinterval having a duration of between 1 millisecond and 4 milliseconds.

19. The method of claim 15, wherein:
the clamping includes switching clamping transistors to a conductive state to connect the circuit nodes to the clamp voltage; and
the unclamping includes switching the clamping transistors to a nonconductive state to disconnect the circuit nodes from the clamp voltage.

20. A system for combined electromyography (EMG) measurements of a target anatomy and stimulation of the target anatomy, the system comprising:
a garment configured to be worn on the target anatomy and including electrodes arranged to electrically contact skin of the target anatomy when the garment is worn on the target anatomy;
a stimulator configured to deliver transcutaneous electrical stimulation to the target anatomy via the electrodes of the garment;
stimulator isolation circuitry configured to switch between electrically connecting the stimulator with the electrodes of the garment and electrically disconnecting the stimulator from the electrodes of the garment;
EMG circuitry configured to receive analog EMG signals from the electrodes of the garment and including analog amplifiers configured to amplify the analog EMG signals to form amplified analog EMG signals and analog-to-digital converters configured to digitize the amplified analog EMG signals to form digitized EMG data;
EMG isolation circuitry configured to switch between electrically connecting the EMG circuitry with the electrodes of the garment and electrically disconnecting the EMG circuitry from the electrodes of the garment;
control circuitry configured to cyclically switch between:
(i) stimulation time intervals during which the stimulator isolation circuitry electrically connects the stimulator with the electrodes of the garment and the EMG isolation circuitry electrically disconnects the EMG circuitry from the electrodes of the garment, and
(ii) EMG time intervals during which the stimulator isolation circuitry electrically disconnects the stimulator from the electrodes of the garment and the EMG isolation circuitry electrically connects the EMG circuitry with the electrodes of the garment; and
clamping transistors electrically connected at circuit nodes located between the EMG isolation circuitry and the EMG circuitry, wherein the control circuitry is further configured to cyclically switch between clamped time intervals in which the clamping transistors are conductive to connect the clamped circuit nodes to a clamp voltage and unclamped time intervals in which the clamping transistors are nonconductive;
wherein each clamped time interval is a contiguous time interval that includes a corresponding stimulation time interval and a recovery time subinterval of an EMG time interval immediately following the corresponding stimulation time interval.
